(19) United States
(12) Patent Application Publication
(10) Pub. No.: US 2022/0080093 A1
(43) Pub. Date: Mar. 17, 2022

(54) SIMULTANEOUS ECMO AND CRRT

(71) Applicant: TEL HASHOMER MEDICAL RESEARCH, INFRASTRUCTURE AND SERVICES LTD., Ramat Gan (IL)

(72) Inventor: Alexander KOGAN, Modiin (IL)

(21) Appl. No.: 17/416,508

(22) PCT Filed: Dec. 26, 2019
(86) PCT No.: PCT/IL2019/051414

§ 371 Date: Jun. 20, 2021

(30) Foreign Application Priority Data
Dec. 27, 2018 (IL) 264001

(51) Int. Cl. A61M 1/36; A61M 1/14; A61M 1/34

(52) U.S. Cl. CPC A61M 1/3607; A61M 1/14; A61M 1/3413; A61M 2205/52; A61M 1/3663; A61M 1/3655; A61M 2205/18; A61M 1/3666

(57) ABSTRACT

A controller for controlling an apparatus comprising a blood pump, oxygenator, and blood filtering system connected together by blood flow tubing and to a patient to circulate blood from the patient through the apparatus and back to the patient, to oxygenate and filter the blood, the controller comprising at least one communication interface operable to receive signals generated responsive to monitored parameters characterizing blood flow and/or quality of blood from the patient circulating through the apparatus and configured to: determine values for the monitored parameters based on the monitoring signals; determine whether or not a value determined for a given monitored parameter for blood circulating through the apparatus is within a normative range for the parameter; and generate an alarm to alert an operator of the system if the value is not within the normative range.

### RELATED APPLICATIONS

This application claims benefit under 35 U.S.C. § 119(a)-(d) of Israeli Application 264001, filed Dec. 27, 2018, the disclosure of which is incorporated herein by reference.

### TECHNICAL FIELD

Embodiments of the disclosure relate to an apparatus and system integrating extracorporeal membrane oxygenation and hemodialysis and/or continuous venous hemofiltration.

### BACKGROUND

Extracorporeal membrane oxygenation (ECMO), also known as extracorporeal life support, is an extracorporeal technique for providing prolonged cardiac and respiratory support to a patient whose heart and lungs are unable to provide an adequate amount of gas exchange, similar to a heart-lung machine. It is a lifesaving procedure used in treating neonates, children and adults with severe, reversible, cardiopulmonary failure. Patients undergoing ECMO are generally at high risk of developing acute kidney injury (AKI) and/or fluid overload (FO). To avoid AKI and FO, renal replacement therapy (RRT) is commonly used to maintain fluid balance and metabolic control. RRT options during ECMO include peritoneal dialysis, intermittent hemodialysis, and continuous RRT (CRRT). The two most common methods to provide CRRT during ECMO are via the use of an in-line hemofilter or via a traditional CRRT device connected to an ECMO device.

ECMO and current CRRT devices operate at respectively different rates of blood flow and different blood pressures. Whereas ECMO typically operates in a relatively high range of blood flow (2-5 L/min), CRRT typically operates in a relatively low range of blood flow (100-300 mL/min). Efficient coupling of ECMO with CRRT so that they both function substantially simultaneously to provide a patient with oxygenation and removal of blood toxins and excess fluid is complex.

### SUMMARY

An aspect of an embodiment of the disclosure relates to providing an integrated blood oxygenation and purification apparatus, hereinafter also referred to as an “OXYPURE” apparatus, or simply OXYPURE, for simultaneously providing a patient with extracorporeal membrane oxygenation (ECMO) and continuous renal replacement therapy (CRRT). In an embodiment OXYPURE comprises a blood oxygenator and a blood filtering system, that removes excess water, solutes, and toxins from the blood. The blood filtering system receives oxygenated blood from the oxygenator for filtering via an input shunt connected to an output tube of the oxygenator through which a flow of oxygenated blood is delivered to the patient. Filtered blood that exits the blood filtering system is returned to an inlet of a blood pump that drains blood from the patient and pumps the blood into the oxygenator. A plurality of blood flow and quality sensors monitor blood flow parameters in blood from the patient circulating in OXYPURE. The sensors transmit monitoring signals responsive to the parameters to a controller that controls components in OXYPURE based on the monitoring signals to provide a desired quantity and quality of blood flow from and to the patient. In an embodiment the controller comprises a database that defines normative, aberrant, and emergency ranges for values of parameters monitored by the sensors. The controller may control components of OXYPURE based on the ranges.

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Unless otherwise indicated, the word “or” in the description and claims is considered to be the inclusive “or” rather than the exclusive or, and indicates at least one of, or any combination of items it conjoins.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure may be used to reference the given feature. Dimensions of features shown in the figures are chosen for convenience and clarity of presentation and are not necessarily shown to scale.

FIG. 1 schematically shows an OXYPURE apparatus operating as a closed system, having a combination of ECMO and CRRT, in accordance with an embodiment of the disclosure;

FIGS. 2A-2C show a Range Table, that lists and defines normative, aberrant, and emergency ranges for parameters that sensors shown in FIG. 1 monitor, in accordance with an embodiment of the disclosure;

FIGS. 3A-3B show a flow diagram of a procedure that a controller in OXYPURE may execute to process monitoring signals based on normative, aberrant, and emergency ranges for values of parameters monitored by sensors comprised in OXYPURE, in accordance with an embodiment of the disclosure;

FIG. 4 schematically shows an OXYPURE apparatus operating as an open system, in accordance with an embodiment of the disclosure; and

FIG. 5 schematically shows a stand-alone blood filtration system, configured to be combined with any of various systems for which blood purification may be advantageous, in accordance with an embodiment of the disclosure; and

### DETAILED DESCRIPTION

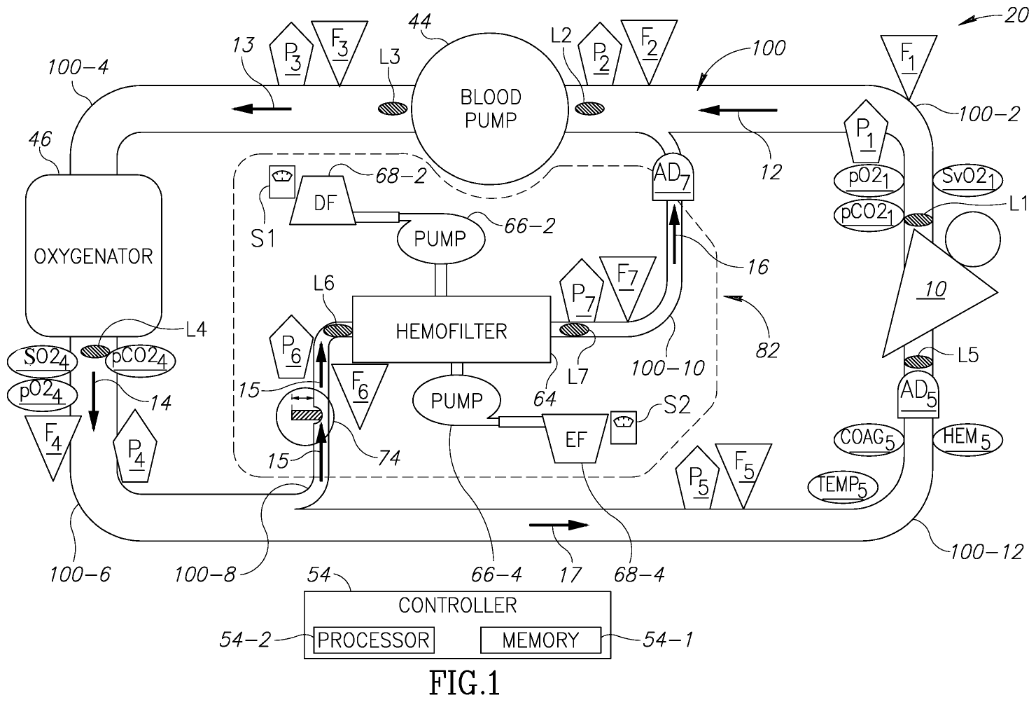

FIG. 1 schematically shows an OXYPURE apparatus 20 operating in a closed system configuration to oxygenate and filter blood from a patient 10, in accordance with an embodiment of the disclosure. In the closed system configuration blood from patient 10 enters OXYPURE 20 directly from the patient's cardiovascular system and flows substantially only within the components and tubing of OXYPURE and the cardiovascular system of patient 10.

OXYPURE 20, optionally comprises a blood pump 44, a blood oxygenator 46, and a blood filtering system 82 comprising a hemofilter 64 that are connected to patient 10 and to each other by blood flow tubing 100. Different sections of blood flow tubing 100 are referenced by the number 100 followed by a dash and a distinguishing number. For example, hemofilter 64 is connected to OXYPURE 20 by an input shunt tubing labeled 100-8 and an output shunt tubing 100-10. A controller 54 controls components of OXYPURE 20, for example blood pump 44 and blood filtering system 82, responsive to monitoring signals communicated by the components wirelessly or by wire to the controller, and monitoring signals communicated wirelessly or by wire to the controller by at least one blood flow sensor that OXYPURE 20 comprises. A monitoring signal received by controller 54 comprises a signal generated responsive to a parameter relevant to the functioning of OXYPURE 20, which signal may be processed to determine a value for the parameter.

Blood pump 44 operates to circulate blood from the patient through tubing 100 and the various components of OXYPURE 20 and back to the patient and may by way of example, be any one of a centrifugal, peristaltic, or roller pump and drains. Oxygenator 46 serves as an artificial lung for patient 10. The oxygenator receives blood schematically represented by an arrow 12, that blood pump 44 drains from the patient and removes carbon dioxide from, and adds oxygen to the blood. The oxygenator typically comprises a semi-permeable membrane, which allows passage of molecular oxygen and carbon dioxide through the membrane while preventing transfer of water through the membrane. Oxygenator 46 may be part of a heart-lung machine or of an ECMO.

Hemofilter 64 filters a portion of blood oxygenated by the oxygenator that is shunted to the hemofilter via an input shunt tubing 100-8 to remove waste products and excess water from the shunted blood. In accordance with an embodiment of the disclosure, the filtered blood, schematically represented by an arrow 16, leaves hemofilter 64 and is returned via an output shunt tubing 100-10 to join blood, represented by arrow 12, flowing from patient 10 into blood pump 44. An amount of blood shunted to hemofilter 64 may be controlled by a blood flow regulator 74. Blood flow regulator 74 may comprise a pinch valve, an external pipe screw clamp, a controllable aperture, optionally a camera-shutter type aperture, and is by way of example, schematically represented as a pinch valve in FIG. 1. In an embodiment, blood flow regulator 74 may be operated to substantially completely block blood flow through input shunt tubing 100-8.

Hemofilter 64 of blood filtering system 82 may be a hemodialysis filter, comprising a semipermeable membrane through which water and waste products are removed from the blood to a flow of a dialysate. The waste product may comprise for example, end-products of nitrogen metabolism, such as urea, creatinine, uric acid, and/or high molecular weight solutes. A dialysate pump 66-2, in the blood filtering system may pump and control flow of dialysate to the filter from a container 68-2. A drainage pump 66-4, of the blood filtering system may operate to drain effluent fluid out of hemofilter 64 and into a receptacle 68-4. Weight detectors S1 and S2 may operate to monitor weight respectively of dialysate in container 68-2 and effluent in receptacle 68-4. Pumps 66-2 and 66-4 and weight detectors S1 and S2 may transmit hemofilter monitoring signals relevant to their respective performances, and functionality provided by hemofilter 64 to controller 54. The controller may control the pumps based on the received hemofilter monitoring signals. In an embodiment, controller 54 may control pumps 66-2 and 66-4 and blood flow regulator 74 to provide hemodialysis intermittently for successive periods of, optionally 8 to 12 hours, or alternatively, continuously until saturation of hemofilter 64. Saturation typically occurs following use for a period of about 72 hours or longer. Optionally, hemofilter 64 may provide blood purification through a combination of hemofiltration and dialysis. Hemodialysis rate may be increased by providing OXYPURE 20 with additional pumps and/or providing blood filtering system 82 with additional hemofilters.

In an embodiment the at least one sensor optionally comprises blood pressure, blood flow, and/or blood gas sensors, and/or air bubble detectors operable to acquire measurements of blood flow parameters at various locations along blood flow tubing 100. Locations for which the sensors optionally acquire measurements of blood pressure, flow and/or gases are schematically indicated by shaded ellipses labeled L1, L2, . . . L7. Ellipses labeled L1 and L5 schematically indicate and refer to regions substantially at locations, at which OXYPURE 100 draws blood from and returns blood to patient 10 respectively. L1 and L5 may be referred to respectively as a patient output and a patient input. Similarly, L2 and L3 refer respectively to blood pump input and blood pump output at which blood enters blood pump 44 and blood exits the blood pump respectively. L4 refers to a region of tubing 100 at an output of oxygenator 46 and L6 and L7 refer respectively to regions along tubing 100 at which measurements may be acquired that are ascribable to a blood flow input and a blood flow output of hemofilter 64.

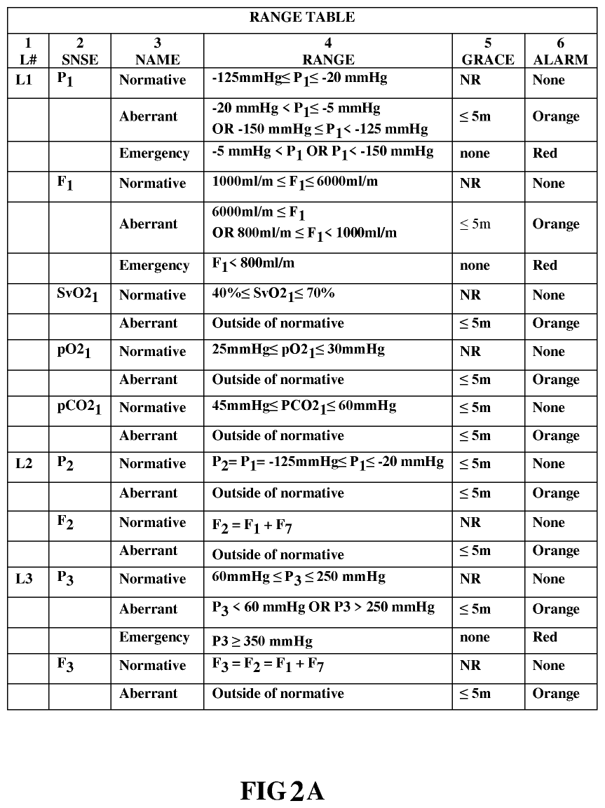

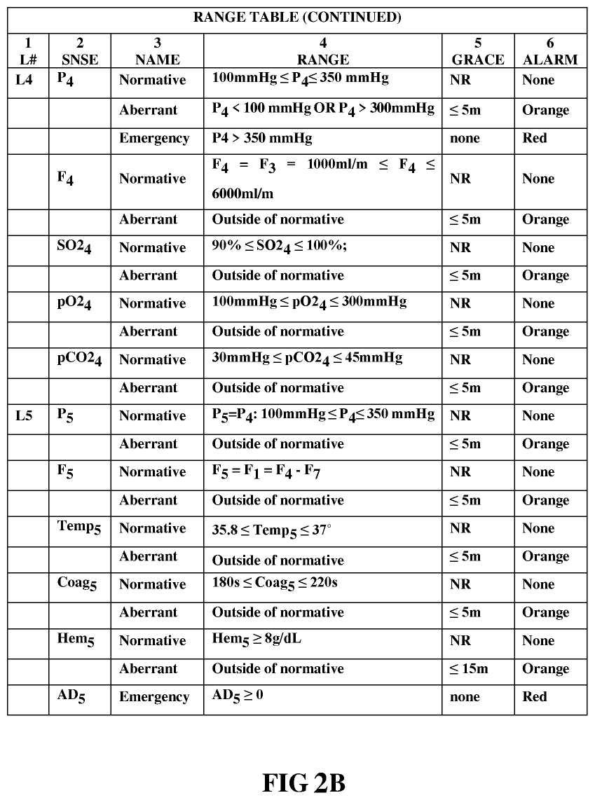

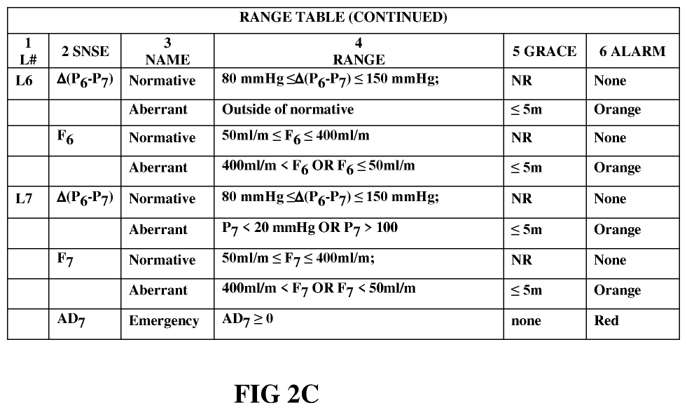

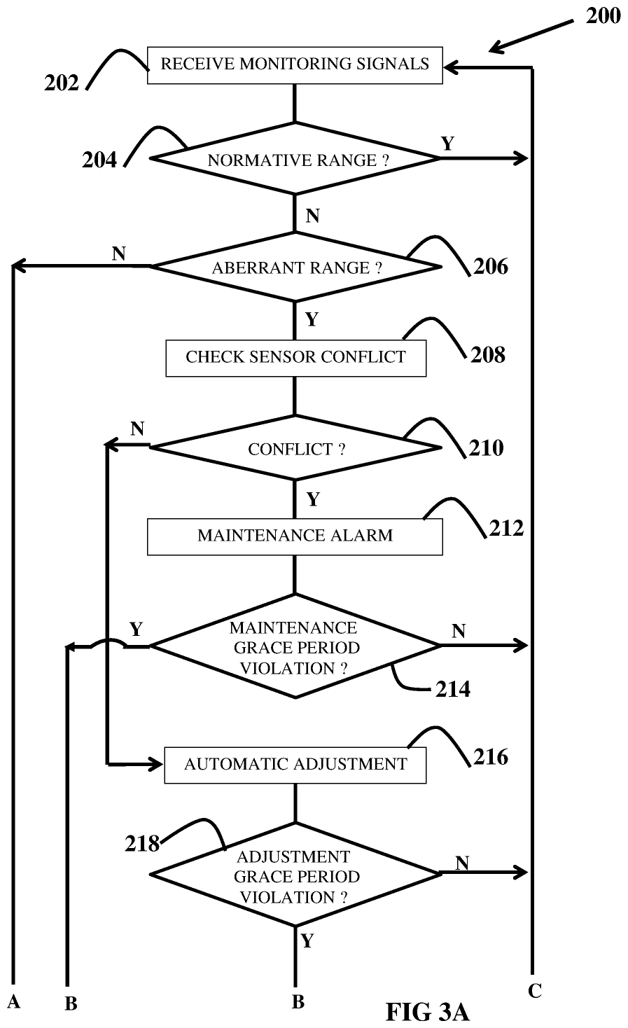

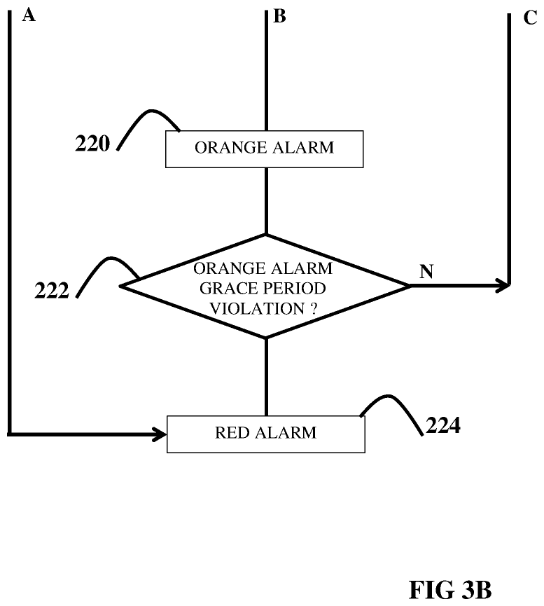

Blood pressure sensors in FIG. 1, and in FIGS. 2 and 3 that are discussed below, are schematically represented by pentagonal icons labeled P₁, P₂, . . . , P₇, where the subscript refers to the location L1, L2, . . . , L7 for which pressure measurements are obtained by the blood pressure sensors. The blood pressure sensors may be referenced generically by the letter “P” common to all their respective labels. Similarly, blood flow sensors are schematically represented by triangular icons, labeled F₁, F₂, . . . , F₇ and may be referred to generically by their shared label letter “F”. Sensors other than blood flow and pressure sensors may be schematically represented by ellipses, each ellipse labeled with a subscripted acronym or short title. The acronym or title designates the type of measurement that the sensor represented by the ellipse provides and the subscript a location L1, L2, . . . , L7 for which the measurement is provided. Blood gas sensors that provide measures of venous blood O₂ (Oxygen) saturation, partial O₂ pressure, and partial CO₂ (carbon dioxide) pressure for patient output L1, are labeled SvO2₁, pO2₁, and pCO2₁ respectively. Blood gas sensors that provide measures of blood O₂ (Oxygen) saturation, partial O₂ pressure, and partial CO₂ pressure for oxygenator output L4, are labeled SO2₄, pO2₄, and pCO2₄ respectively. Measurements of blood temperature, coagulation rate, and hemoglobin performed by sensors optionally for patient input L5, are respectively labeled Temp₅, COAG₅, and HEM₅. An air bubble detector operable to detect air bubbles at location L5 and, optionally, also to stop blood flow to patient 10 upon detection of air bubbles is labeled AD₅. An air bubble detector AD₇, is operable to detect air bubbles at location L7 and also, optionally, to stop blood flow 16 from hemofilter 64 from entering blood pump 44 upon detection of air bubbles. Each of the OXYPURE sensors and/or air bubble detectors may communicate with controller 54 to transmit monitoring signals to controller 54 and/or receive signals from the controller by any of various wire and/or wireless communications channels.

Values that a given sensor provides for a parameter that the sensor monitors may be represented by the same label that labels the icon representing the sensor in FIG. 1. For example, pressure sensor P₁ provides pressure values P₁ for pressure at location L1, blood flow sensor F₃ provides values F₃ for blood flow at location L3, and CO₂ partial pressure monitor pCO2₄. provides values pCO2₄ for CO₂ partial pressure at location L4.

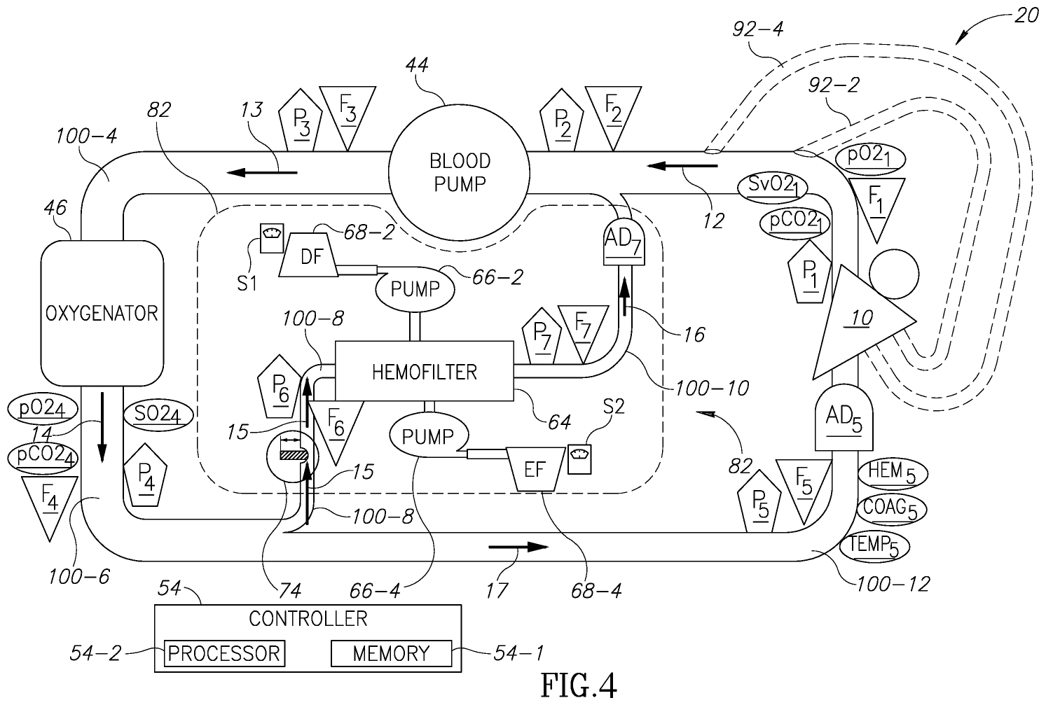

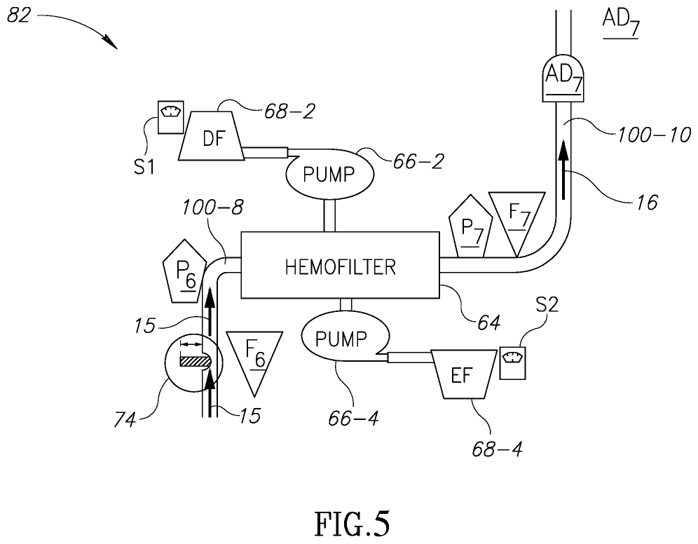

It is noted that whereas FIG. 1, and FIGS. 4 and 5, show a particular number and configuration of components and sensors, embodiments of the disclosure are not limited to the number and configuration of components and sensors shown. For example, an OXYPURE in accordance with an embodiment of the disclosure similar to OXYPURE 20, may comprise more than one sensor for providing a same measurement at substantially a same location in OXYPURE. Monitoring signals from the additional sensors are optionally used to recognize and reduce incidents of erroneous measurements and improve reliability of OXYPURE functioning. An OXYPURE may comprise sensors that measure blood and blood flow parameters at an input (not labeled) to oxygenator 46 at which blood from blood pump 44 enters the oxygenator in addition to sensors at output L4 of the oxygenator. OXYPURE apparatus 20 may also comprise a device (not shown) operable to intravenously infuse an anticoagulant solution into patient. The anticoagulant solution may, by way of example, comprise any one or any combination of more than one of heparin, citrate, bivalirudin or any other anti-coagulant substance suitable for use with extracorporeal apparatuses. The device that delivers the anti-coagulant solution may comprise an anti-coagulant pump, which may be a single unit (systemic anti-coagulation, e.g., heparin), or alternatively it may be part of a more complex infusion system with multiple anti-coagulation pumps. In an embodiment of the disclosure, OXYPURE apparatus 20 may comprise a pump to infuse antagonists to anti-coagulants to blood from patient 10, which may be necessary in case anti-coagulation is only needed in a very localized part of the circuit.

In operation OXYPURE pump 44 drains blood from patient 10 through a pump input tubing section 100-2 via a suitable drainage cannula (not shown) optionally located in the inferior vena cava of the patient (not shown). The blood pump drains the blood at a relatively low pump inlet pressure, in a normative pressure range between about −125 mmHg (millimeters of mercury) and about −20 mmHg at patient output L1, as optionally measured by pressure sensor P₁, and at a normative blood flow rate at L1 between about 1,000 ml/m (milliliters per minute) and about 6,000 ml/m, as optionally measured by flow sensor F₁.

Drop in blood pressure between location L1 as measured by P₁ and blood pressure at location L2 as measured by P₂ is expected under normal operating conditions to be negligible, as resistance to blood flow through tubing 100-2 is expected to be very low. Fluid flow rate as measured by F₂ at L2 is however expected to be greater than fluid flow rate as measured by F₁ at L2, because as noted above and discussed below, blood flow rate at L2 is a sum of blood flow schematically represented by arrow 12 through tubing 100-2 and blood flow schematically represented by arrow 16 through shunt tubing 100-10 that delivers blood from hemofilter 64 to L2. Blood pump 44 pumps blood 12 that enters blood pump 44 from patient 10 via tubing section 100-2 and blood 16 from hemofilter 64 via a tubing section 100-4 into oxygenator 46 at a relatively high, pump outlet pressure at pump outlet L3. Normative pump outlet pressure range at L3 may be between about 60 mm Hg and about 250 mm Hg. Blood flowing from blood pump 44 to oxygenator 46 is schematically represented by an arrow 13.

Following oxygenation in oxygenator 46, oxygenated blood schematically represented by an arrow 14 leaves the oxygenator substantially at location L4 to flow via oxygenator output tubing 100-6 towards patient 10. Blood pressure at L4 as measured by sensor P₄ has a normative pressure range between about 100 mmHg and about 350 mmHg. Blood flow at L4 as measured by F₄ has a normative range between about 1000 ml/m and about 6000 ml/m. Optionally, as shown in FIG. 1, O₂ saturation of oxygenated blood at L4 is measured by monitor SO2₄ and has a normative oxygen saturation range between about 90% and about 100%. Partial pressure of O₂ at L4 is measured by monitor pO2₄ and has a normative O₂ partial pressure range between about 100 mmHg and about 300 mmHg. Partial pressure of CO₂ at L4 is measured by monitor pCO2₄ and has a normative CO₂ partial pressure range between about 30 mmHg and about 45 mmHg.

A portion, optionally between about 5% and 10%, of oxygenated blood 14 flowing in oxygenator output tubing 100-6 is shunted into blood filtering system 82 and its hemofilter 64 via input shunt tubing 100-8. Blood flowing in input shunt tubing 100-8 to hemofilter 64 is represented by arrows 15. Pressure drop across hemofilter 64 downstream of blood flow regulator 74 and upstream of air bubble detector AD₇ as determined from measurements provided by P₆ and P₇ respectively has a normative pressure range between about 80 mmHg and about 150 mmHg. Blood flow at L6 and L7 as measured by respectively by F₆ and F₇ has a normative range between about 50 ml/m and about 400 ml/m.

A portion, optionally between about 90% to about 95%, of the oxygenated blood flowing in oxygenator output tubing 100-6 that is not shunted to hemofilter 64 flows through output tubing 100-12 to return to patient 10 via a return cannula (not shown) inserted, optionally, into the patient's femoral artery. At patient input location L5 blood pressure is measured by sensor P₅ and is expected to be the same as blood pressure at L4 and have a normative pressure range between about 100 mmHg and about 350 mmHg. However, blood flow at L5 as measured by F₅ is expected to be equal to blood flow at L4 as measured by P₄ minus flow at L6 as measured by F₆ and have a normative range that is the same as the normative range for blood at L1, which is between about 1000 ml/m and about 6000 ml/m. Optionally, as noted above and as schematically shown in FIG. 1, OXYPURE 20 sensors TEMP₅, COAG₅ and HEM₅ acquire measurements for temperature, coagulation and hemoglobin at L5. Temperature at L5 may have a normative range between about 35.8° C. and 37° C. Coagulation at L5 has a normative range between about 180 s (seconds) and about 220 s and hemoglobin at L5 has a normative range greater than about 8 g/dL (grams per deciliter).

In an embodiment controller 54 processes monitoring signals that the controller receives from sensors comprised in OXYPURE 20 to control components in OXYPURE 20 to provide a desired flow and quality of blood from and to patient 10, and to determine when to generate alarms that alert operators to conditions of OXYPURE and/or patient 10 that require operator attention and/or intervention. The controller comprises or has access to any of various physical and/or virtual electronic and/or optical memories and processors for storing and using data and executable instructions, collectively referred to as software, that may be required to process the received monitoring signals and enable functionalities with which the controller is tasked to provide advantageous functioning of OXYPURE 20. The memory and/or processor may be stand alone, distributed and/or cloud based. The software may be of any suitable format and architecture and may for example be rule based and/or comprise machine learning algorithms and/or artificial intelligence (AI).

By way of example, controller 54 is schematically shown comprising a memory 54-1 and a processor 54-2 respectively representing at least one memory and at least one processor that the controller may comprise and/or to which the controller may have access. Memory 54-1 may have any electronic and/or optical circuitry suitable for storing data and/or computer executable instructions and may, by way of example, comprise any one or any combination of more than one of a flash memory, random access memory (RAM), read only memory (ROM), and/or erasable programmable read-only memory (EPROM). Memory 54-1 may be a distributed cloud-based memory. Processor 54-2 may comprise any processing and/or control circuitry known in the art and may by way of example comprise any one or any combination of more than one of a microprocessor, an application specific circuit (ASIC), field programmable array (FPGA) and/or system on a chip (SOC). Processor 54-2 may be a distributed cloud-based memory. Controller 54 software may comprise an artificial intelligence (AI).

In an embodiment, controller 54 comprises and/or has access to any of appropriate audio and/or display devices (not shown), hereinafter collectively referred as alarm devices, controllable by the controller to generate alarms that alert an operator to a condition of OXYPURE 20 that the controller determines requires operator attention and/or intervention. Alarm devices may by way of example comprise dedicated speakers and/or display screens and/or speakers and/or display screens comprised in any of various mobile smart devices such as for example mobile phones, tablets, augmented reality devices, and/or laptops.

In an embodiment, memory 54-1 is stored with a database that comprises data defining the normative ranges noted above for values of parameters monitored by sensors in OXYPURE 20 and for which the sensors generate monitoring signals transmitted to the controller. Optionally, for a given sensor the operating database comprises in addition to data defining a normative range for values of parameters monitored by the sensor, data defining a range, optionally referred to as an aberrant range, for values of the parameter that are considered to be moderate deviations from the normative range. In accordance with an embodiment, an aberrant range for values of the given sensor is associated with an alarm, also referred to as an “orange alarm”, defined by an orange alarm profile and optionally a grace period. The orange alarm profile defines stimuli, optionally audio and/or visual stimuli, which controller 54 generates to attract attention of an operator to detection of an aberrant value for the parameter monitored by the given sensor. The orange alarm grace period determines a period of time that is allotted for effecting action that addresses and remediates a cause of the orange alarm from a time at which the orange alarm is activated.

In an embodiment of the disclosure in the event of an orange alarm, controller 54 may be configured to automatically address and attempt rectify a situation that caused the controller to generate the orange alarm. Depending, on the parameter for which the controller generates the orange alarm the controller may attempt to control at least one or any combination of more than one of pumping speed of blood pump 44, oxygen pressure in oxygenator 46, or flow regulator 74. If OXYPURE 20 comprises a source of supplemental oxygen, for example, an oxygen mask and ventilator (not shown) attached to patient 10, the controller may additionally or alternatively adjust an amount of oxygen provided to the patient 10 to address the cause of the alarm. Alternatively, or additionally an orange alarm may be intended to alert an operator to intervene to address the cause of the alarm.

Optionally, the operating database comprises data defining a range, referred to as an emergency range, for values of the parameter measured by the given sensor that are considered extreme and to require immediate operator intervention to rectify a cause of the extreme values. In accordance with an embodiment, the emergency range is associated with an alarm, referred to as a “red alarm”, defined by a red alarm profile, which controller 54 generates in response to a parameter having a value within the emergency range. The red alarm profile defines particularly arousing stimuli, optionally audio and/or visual stimuli, that controller 54 generates to signal an operator that immediate action is required to rectify the cause of a red alarm. A red alarm may also be generated in the event that the cause of an orange alarm is not corrected before expiration of an orange alarm grace period associated with the orange alarm.

FIGS. 2A, 2B, and 2C show a table, which may be referred to as a Range Table, that lists and defines normative, aberrant, and emergency ranges for parameters that sensors shown in FIG. 1 monitor.

A first column headed “L#”, of the Range Table shows a location L1, L2, . . . , L7 for which sensors given in a second column, headed “SNSE” of the table provide measurements. An empty cell in the columns is assumed to have a value that is the same as that of a first non-empty cell above the empty cell. A third column headed “NAME” gives the name “Normative”, “Aberrant”, or “Emergency” of a range for which a value range is given in a fourth column headed “RANGE” in the Range Table. A fifth column headed “GRACE” gives a grace period by which an action associated with the range named in the same row as the grace period is to be undertaken. For Normative ranges, grace periods may not be relevant and cells in the GRACE column associated with normative ranges are entered with “NR”. Grace periods for Aberrant ranges are entered with appropriate respective time limits in minutes, for example “≤5 m”, by which actions in association with the ranges are to be undertaken, in accordance with an embodiment. Emergency ranges which are considered to require immediate remedial action do not have a grace period and cells in the GRACE column associated with emergency ranges are entered with “none”. A given cell in a last column, headed “ALARM”, in the Range Table indicates a type of alarm to be generated if a parameter value falls within the range given in the “RANGE” column for the same row in which the given cell is located. A cell in the RANGE column may have an entry “None”, Orange, or Red to indicate that, no alarm, an Orange alarm, or a Red alarm is to be raised.

FIGS. 3A and 3B show a flow diagram of a procedure 200 in accordance with which controller 54 and processor 54-2 optionally process monitoring signals that the controller receives from a sensor in OXYPURE 20.

In a block 202, controller 54 receives a stream of monitoring signals for a given parameter monitored by a given sensor. In a decision block 204 processor 54-2 processes the signals to determine a stream of values for the given parameter corresponding respectively to the received monitoring signals. If the determined values in the stream of values are within the normative range for the parameter processor 54-2 returns to block 202 to engage in normal reception and processing of monitoring signals for the given parameter.

If in decision block 204 processor 54-2 determines that a sequence of values for the given parameter are not within the normative range, processor 54-2 optionally advances to a decision block 206 to determine if the values in the sequence are within the aberrant range of values for the parameter. If the values are determined not to be in the aberrant range, the values are in the emergency range and processor 54-2 optionally proceeds to a block 224 (FIG. 5B) to generate an emergency red alarm.

If on the other hand, in block 204 the processor determines that values in the sequence of values are in the aberrant range, the processor optionally proceeds to a block 208 to check whether or not the values in the sequence conflict with values for parameters provided by other sensors in OXYPURE 20. A value for a given parameter conflicts with values of other parameters if the value for the given parameter and the values for the other parameters are related by a known relationship, and the value of the given parameter conflicts with the relationship while the values for the other parameters agree with the relationship. For example, from continuity of blood flow under normal operation of OXYPURE, it is expected that for blood flow, values F₂, F₃, F₄ satisfy a constraint F₂=F₃=F₄ (FIG. 1). If F₂=F₄ but F₃≠F₄, then F₃ is in conflict with both F₂ and F₄ and it is expected that sensor F₃ is subject to a malfunction and provided an incorrect value for blood flow F₃. (As noted above values that a given sensor provides for a parameter that the sensor monitors may be referenced by the same reference label that labels the icon representing the sensor in FIG. 1). In an embodiment, in a decision block 210 if the sequence of parameter values determined from the stream of monitoring signals received in block 202 and checked for conflict in block 208 is in conflict, processor 54-2 optionally proceeds to a block 212. In block 212 the processor generates an alarm, hereinafter also referred to as a maintenance alarm, to alert an operator of OXYPURE 20 to undertake a maintenance operation to correct for a possible malfunction of the given sensor generating the stream of monitoring signals.

In a decision block 214, for a predetermined maintenance grace period, controller 54 optionally continues to receive monitoring signals from the given sensor and processor 54-2 operates to determine if the monitoring signals provide values for the given parameter that are no longer aberrant but instead have returned to being normative. If the values are normative, maintenance has been successfully undertaken and controller 54 returns to block 202 to engage in normal reception and processing of monitoring signals for the given sensor. If on the other hand successful maintenance is not achieved within the maintenance grace period, values for the given parameter have not returned to being normative, the maintenance grace period is violated, and processor 54-2 optionally proceeds to a block 220 (FIG. 5B) to generate an orange alarm.

In a decision block 222 processor 54-2 optionally determines whether or not within an orange alarm grace period for the given parameter from a time at which the orange alarm was initiated as a result of maintenance monitoring signals from the given sensor have reverted to being normative. If the monitoring signals are determined to be normative, the orange alarm grace period is not violated and processor 54-2 quenches the orange alarm and returns to normal operation in block 202. If on the other hand the orange alarm grace period is determined to be violated, processor 54-2 optionally proceeds to block 224 and generates a red alarm to alert the OXYPURE operator to an emergency situation and to undertake immediate action to return OXYPURE 20 to normative ranges.

If in block 210 the values of the given parameter in the sequence of values are determined not to be in conflict, processor 54-2 may proceed to a block 216 to cause controller 54 to attempt to automatically adjust components of OXYPURE 20 to restore normative values for the given parameter and normative functioning of OXYPURE 20 with a predetermined adjustment grace period. In a block 218 if automatic adjustment has been successful, controller 54 returns to block 202 to resume normal operation. If on the other hand automatic adjustment has not been successful and the predetermined adjustment grace period violated, processor 54-2 may proceed to block 224 and generate a red alarm.

FIG. 4 schematically shows OXYPURE 20 operating in an open system configuration in accordance with an embodiment of the disclosure. In the open system configuration, blood from patient 10 is not constrained to flow only within the components and tubing of OXYPURE and the cardiovascular system of patient 10. Some of the patient's blood, as might result from damage to a blood vessel, may leak out from the patient's cardiovascular system to pool in a body cavity or cavities of the patient. To maintain integrity of the patient blood volume, at least one suction tube may be connected to OXYPURE 20 to suction blood leaking out of the patient's cardiovascular system into OXYPURE 20 and thereby return the blood to the patient's cardiovascular system. By way of example, OXYPURE 20 is schematically shown in FIG. 2 having two suction tubes 92-2 and 92-4 delineated in dashed lines connected to pump input tubing 100-2 to suction blood leaking from patient 20 and into blood pump 44.

Whereas in FIGS. 1 and 4 blood filtering system 82, indicated by a dashed line, is integrated as part of OXYPURE 20 the blood filtration system may be a modular unit configured to be coupled as an add-on to any of various machines that might be used to assist in oxygenation of a patient's blood, to provide blood filtering. In an embodiment of the disclosure, blood filtering system 82 may be integrated into any one of a heart-lung machine for cardio-pulmonary bypass during cardiac surgery, or an ECMO machine for long-term cardio-pulmonary support. Optionally, the blood filtering system may be used on its own to assist a patient in maintaining healthy blood. FIG. 3 schematically shows blood filtering system 82, in accordance with an embodiment, as a standalone unit configured to be connected, by way of example, to an ECMO apparatus or to a patient.

There is therefore provided according to an embodiment of the disclosure a controller for controlling an apparatus to oxygenate and filter blood from a patient, the apparatus comprising a blood pump, oxygenator, and blood filtering system, the controller comprising: (i) at least one communication interface operable to receive wireless and/or wire monitoring signals generated in response to monitored parameters characterizing blood flow and/or quality of blood from the patient circulating through the apparatus at locations in and outside of the blood filtering system; (ii) a memory comprising a database having data that defines normative ranges for values of the monitored parameters; and (iii) a processor comprising computer executable instructions executable to: (a) determine values for the monitored parameters based on the monitoring signals; (b) determine whether or not a value determined for a given monitored parameter for blood circulating through the apparatus is within the normative range for the parameter; and (c) generate an alarm to alert an operator of the system if the value is not within the normative range. The apparatus is connected together by blood flow tubing and connected to a patient to circulate blood from the patient through the apparatus and back to the patient.

In an embodiment, the database has data that defines an aberrant range for values of the monitored parameters that lie outside of the normative range.

In an embodiment, the executable instructions comprise instructions executable to determine whether or not the value determined for the given parameter lies in the aberrant range. Optionally, the executable instructions comprise instructions executable to determine whether the aberrant value for the given parameter conflicts with a normative value of at least one other monitored parameter.

In an embodiment, the database comprises a maintenance grace period, that defines a time period for effecting successful maintenance from a time at which the conflict is determined. In an embodiment, the database comprises an adjustment grace period that defines a time period for automatically effecting successful automatic adjustment of the apparatus from a time at which the conflict is determined.

In an embodiment, the executable instructions comprise instructions executable to generate a maintenance alarm on an alarm device to alert an operator of the apparatus to the determined conflict and to undertake a maintenance operation to correct for a possible malfunction of the given sensor. Optionally, the executable instructions comprise instructions executable to generate an aberrant alarm on an alarm device to alert an operator of the apparatus that operator intervention is required if successful sensor maintenance is not achieved within the maintenance grace period.

In an embodiment, the executable instructions comprise instructions executable to automatically adjust the apparatus to attempt to cause values for the given parameter to revert to normative values if the value for the given parameter does not conflict with a value of at least one other monitored parameter.

In an embodiment, the executable instructions comprise instructions executable to generate an aberrant alarm on an alarm device to alert an operator of the apparatus that operator intervention is required to remediate a cause of the aberrant value if successful automatic adjustment is not achieved within the adjustment grace period.

In an embodiment, the database comprises an aberrant grace period and the executable instructions comprise instructions executable to generate an emergency alarm to alert an operator of the apparatus that an emergency exists with respect to functioning of the apparatus and/or wellbeing of the patient and that immediate emergency intervention by operator is required if successful remediation is not achieved within the aberrant grace period.

In an embodiment, the database has data that defines an aberrant range for values for the monitored parameters that lie in a limited range outside of the normative range for which automatic or operator intervention within a predetermined grace period is required to rectify a cause of the aberrant values. In a further embodiment, the database has data that defines an emergency range for values of the monitored parameters that lie in a limited range outside of the normative range and for which immediate intervention by an operator of the apparatus is required to rectify a cause of the extreme values.

There is further provided according to an embodiment of the disclosure an apparatus to oxygenate and filter the blood, the apparatus comprising: (i) a blood pump, an oxygenator for oxygenating blood, and blood filtering system, connected together by blood flow tubing and configured to connect to a patient's cardiovascular system to drain blood from the patient and circulate the blood through the apparatus and back to the patient; and a controller as described herein the disclosure.

In an embodiment, the apparatus comprises a blood flow tubing input shunt that shunts a portion of the blood oxygenated by the oxygenator to the blood filtering system. Optionally, the apparatus comprises a blood flow tubing output shunt that returns blood shunted from the oxygenated blood after passing through the blood filtering system to flow to the oxygenator.

In an embodiment, the apparatus comprises a blood flow output shunt that delivers blood flowing from the blood filtering system to join with blood drained from the patient substantially at an input of the blood pump to be pumped to the oxygenator. Optionally the apparatus comprises a blood flow output shunt that delivers blood flowing from the blood filtering system to join with blood pumped out of the oxygenator to the oxygenator.

In an embodiment, the apparatus comprises a blood flow regulator, for example a pinch valve, for controlling an amount of blood shunted to the blood filtering system from the blood oxygenated blood. In an embodiment, the blood flow regulator is coupled to the input shunt.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the disclosure in the present application are provided by way of example and are not intended to limit the scope of the disclosure. The described embodiments comprise different features, not all of which are required in all embodiments of the disclosure. Some embodiments utilize only some of the features or possible combinations of the features. Variations of embodiments of the disclosure that are described, and embodiments of the disclosure comprising different combinations of features noted in the described embodiments, will occur to persons of the art. The scope of the disclosure is limited only by the claims.

1. A controller for controlling an apparatus comprising a blood pump, oxygenator, and blood filtering system connected together by blood flow tubing configured to circulate blood from a patient through the apparatus and back to the patient, to oxygenate and filter the blood, the controller comprising:
at least one communication interface operable to receive wireless and/or wire monitoring signals generated responsive to monitored parameters characterizing blood flow and/or quality of blood from the patient circulating through the apparatus at locations in and outside of the blood filtering system;
a memory comprising a database having data that defines normative ranges for values of the monitored parameters; and
a processor comprising computer executable instructions executable to:
determine values for the monitored parameters based on the monitoring signals;
determine whether or not a value determined for a given monitored parameter for blood circulating through the apparatus is within the normative range for the parameter; and
generate an alarm if the value is not within the normative range.

2. The controller according to claim 1 wherein the database comprises data that defines an aberrant range for values of the monitored parameters that lie outside of the normative range.

3. The controller according to claim 2 wherein the executable instructions comprise instructions executable to determine whether or not the value determined for the given parameter lies in the aberrant range.

4. The controller according to claim 3 wherein the executable instructions comprise instructions executable to determine whether the aberrant value for the given parameter conflicts with a normative value of at least one other monitored parameter.

5. The controller according to claim 4 wherein the executable instructions comprise instructions executable to generate a maintenance alarm on an alarm device to alert an operator of the apparatus to the determined conflict and to undertake a maintenance operation to correct for a possible malfunction of the given sensor.

6. The controller according to claim 5 wherein the database comprises a maintenance grace period, that defines a time period for effecting successful maintenance from a time at which the conflict is determined.

7. The controller according to claim 6 wherein the executable instructions comprise instructions executable to generate an aberrant alarm on an alarm device to alert an operator of the apparatus that operator intervention is required if successful sensor maintenance is not achieved within the maintenance grace period.

8. The controller according to claim 4, wherein the executable instructions comprise instructions executable to automatically adjust the apparatus to attempt to cause values for the given parameter to revert to normative values if the value for the given parameter does not conflict with a value of at least one other monitored parameter.

9. The controller according to claim 8 wherein the database comprises an adjustment grace period that defines a time period for automatically effecting successful automatic adjustment of the apparatus from a time at which the conflict is determined.

10. The controller according to claim 9 wherein the executable instructions comprise instructions executable to generate an aberrant alarm on an alarm device to alert an operator of the apparatus that operator intervention is required to remediate a cause of the aberrant value if successful automatic adjustment is not achieved within the adjustment grace period.

11. The controller according to claim 10 wherein the database comprises an aberrant grace period and the executable instructions comprise instructions executable to generate an emergency alarm to alert an operator of the apparatus that an emergency exists with respect to functioning of the apparatus and/or wellbeing of the patient and that immediate emergency intervention by operator is required if successful remediation of the aberrant value is not achieved within the aberrant grace period.

12. The controller according to claim 1 wherein the database has data that defines an aberrant range for values for the monitored parameters that lie in a limited range outside of the normative range for which automatic or operator intervention within a predetermined grace period is required to rectify a cause of the aberrant values.

13. The controller according to claim 12 wherein the database has data that defines an emergency range for values of the monitored parameters that lie in a limited range outside of the normative range and for which immediate intervention by an operator of the apparatus is required to rectify a cause of the extreme values.

14. An apparatus comprising:
a blood pump, an oxygenator for oxygenating blood, and blood filtering system connected together by blood flow tubing and configured to connect to a patient's cardiovascular system to drain blood from the patient and circulate the blood through the apparatus and back to the patient, to oxygenate and filter the blood; and
a controller in accordance with claim 1.

15. The apparatus according to claim 14 and comprising a blood flow tubing input shunt that shunts a portion of the blood oxygenated by the oxygenator to the blood filtering system.

16. The apparatus according to claim 15 and comprising a blood flow tubing output shunt that returns blood shunted from the oxygenated blood after passing through the blood filtering system to flow to the oxygenator.

17. The apparatus according to claim 16 and comprising a blood flow output shunt that delivers blood flowing from the blood filtering system to join with blood drained from the patient substantially at an input of the blood pump to be pumped to the oxygenator.

18. The apparatus according to claim 16 and comprising a blood flow output shunt that delivers blood flowing from the blood filtering system to join with blood pumped out of the oxygenator to the oxygenator.

19. The apparatus according to claim 14 and comprising a blood flow regulator that controllable to control an amount of blood shunted to the blood filtering system from the oxygenated blood.

20. The apparatus according to claim 19 wherein the blood flow regulator comprises a pinch valve coupled to the input shunt.
